(19) United States
(12) Patent Application Publication
(10) Pub. No.: US 2006/0153797 A1
(43) Pub. Date: Jul. 13, 2006

(54) Tissue material and matrix

(76) Inventors: Susan Kate Bortolotto, Nunawading (AU); Aurora Messina, Eltham (AU); Keren Maree Abberton, Glen Waverley (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/150,538

(22) Filed: Jun. 10, 2005

(30) Foreign Application Priority Data
Jun. 11, 2004 (AU) 2004903239
Aug. 9, 2004 (AU) 2004904477

(51) Int. Cl. A61K 38/19; A61K 38/37; A61K 38/38; A61K 31/727; A61K 35/34

(52) U.S. Cl. 424/85.1; 424/548; 514/12; 514/56

(57) ABSTRACT

The present invention relates generally to a tissue preparation including tissue cells and extracts thereof useful for promoting or facilitating the growth, development and differentiation of cells and tissues. More particularly, the present invention provides muscle-derived material comprising intact or extracted extracellular matrix and/or cells as well as cytokines, growth factors and other components. The muscle preparations of the present invention resemble basement membrane and are derived from cellular-based material. The muscle preparation may be used in vitro or in vivo as inter alia, a cellular scaffold in various tissue engineering applications and in other cell culture systems for nurturing and enriching a range of cell types including, but not limited to, precursor and stem cells such as pre-adipogenic cells. The muscle preparation is also useful as a base for creams, such as in the cosmetic and topical therapeutic industries and as a matrix or additive in the food industry.

### BACKGROUND OF THE INVENTION

1. Field of the Invention

The present invention relates generally to a tissue preparation including tissue cells and extracts thereof useful for promoting or facilitating the growth, development and differentiation of cells and tissues. More particularly, the present invention provides muscle-derived material comprising intact or extracted extracellular matrix and/or cells as well as cytokines, growth factors and other components. The muscle preparations of the present invention resemble basement membrane and are derived from cellular-based material. The muscle preparation may be used in vitro or in vivo as inter alia, a cellular scaffold in various tissue engineering applications and in other cell culture systems for nurturing and enriching a range of cell types including, but not limited to, precursor and stem cells such as pre-adipogenic cells. The muscle preparation is also useful as a base for creams, such as in the cosmetic and topical therapeutic industries and as a matrix or additive in the food industry.

2. Description of the Prior Art

Bibliographic details of references in the subject specification are also listed at the end of the specification.

Reference to any prior art in this specification is not, and should not be taken as, an acknowledgment or any form of suggestion that that prior art forms part of the common general knowledge in any country.

Basement membranes are thin, continuous sheets that separate epithelium from stroma and surround nerves, muscle fibers, smooth muscle cells and fat cells. Electron microscopic analysis indicates that the components of the basement membranes are a network of filaments which interact to form the membrane. This network, in part, results from the presence of collagen IV molecules which interconnect via intermolecular disulfide bonds (Inoue et al., J Cell Biol, 97:1524-1539, 1983).

The various components of the basement membranes are known to interact with each other. For example, one component of the basement membrane, laminin, binds to collagen IV as well as heparan sulfate proteoglycan.

Basement membrane preparations can provide a physiologically relevant environment which to characterize cell growth, development and differentiation. These preparations are often heterogeneous in composition and in activity. Some preparations, for example, are soluble and lack suitability as a cell matrix (Terranova et al., Cell 22:719-726, 1980).

One preparation derived from Engelbreth Holm-Swarm (EHS) murine sarcoma is a basement membrane-rich matrix sold under the trade name “Matrigel” [Trade Mark, BD Biosciences] and is described by Kleinman et al., Biochem 21:8188-6193, 1982. Matrigel has been a useful product to facilitate cell growth, development and differentiation. However, in some cases, there may be species specific differences in the level of interaction that some cells have with the murine-derived Matrigel which renders this product not suitable for use with non-murine cells such as human cells. It may also illicit immune responses in non-murine hosts.

In accordance with the present invention, a new basement membrane-rich tissue preparation is provided with particularly useful growth, morphological and differentiation promoting activities in a range of cells including human cells.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides cellular and intact and extracted extracellular matrix material which is useful as a scaffold to support the growth, development and differentiation of cells and to support or effect morphological changes to cells. The tissue material is preferably derived from muscle tissue and comprises a preparation comprising basement membrane components. These components comprise one or more of, but not limited to, laminin, collagen I, collagen IV, entactin/nidogen, heparan sulfate proteoglycan as well as one or more of, but not limited to, EGF, bFGF, NGF, PDGF, IGF-1, TGF-β, VEGF and TNF-α. Generally, the tissue material comprises either a cell-based preparation or an intact or extracted extracellular matrix. The intact or extracted cell-free preparation is generally prepared using methods such as urea or SDS extraction or freeze/thawing or freeze drying followed by washing. Cell-based preparations are generally prepared using techniques such mincing, glutaraldehyde fixation and/or freezing in DMSO or other cryo-preservative. Freeze drying does not preserve intact cells but critical point drying does

The tissue material is also conveniently referred to herein as muscle matrix, muscle basement membrane matrix, myomatrix, myotrix, muscle scaffold, myogel and cell culture composition. The term “muscle matrix” is conveniently used for brevity with the understanding that it covers both cell-based and cell-free preparations. A cell free preparation includes intact and extracted extracellular matrix.

The muscle matrix of the present invention has a variety of uses such as in tissue engineering to facilitate the generation of large amounts of tissue for tissue repair, augmentation and/or replacement therapy. The muscle matrix is also useful as a scaffold for engineered tissues such as, but not limited to, muscle and fat. It is also useful as a means to enrich and nurture appropriate pre-adipogenic cells from appropriate stem cell locations. As a research tool, the muscle matrix of the present invention is useful in the study of cell growth, development and differentiation such as of endothelial, epithelial, glial, neuronal, muscle cells and preadipocytes. In the cosmetic and food industries, the muscle preparation is useful as a base for creams and as food additives as well as therapeutically as cellular repair compositions.

The muscle matrix of the present invention is particularly superior to other basement membrane preparations since it induces or otherwise facilitates a wider range of cellular activities and can be applied in a species-conserved way.

Abbreviations used herein are defined in Table 1.

TABLE 1
Abbreviations

| ABBREVIATION | DESCRIPTION |
|---|---|
| bFGF | Basic Fibroblast Growth Factor |
| DMEM | Dulbecco's Modified Essential Medium |
| EGF | Epidermal Growth Factor |
| FCS | Foetal Calf Serum |
| HUVEC | Human umbilical vein endothelial cells |
| IBMX | Isobutylmethylxanthine |
| IGF-1 | Insulin like growth factor-1 |
| NaCl | Sodium chloride |
| NGF | Nerve Growth Factor |
| PDGF | Platelet Derived Growth Factor |
| SDS-PAGE | Sodium dodecylsulphate polyacrylamide |
| | gel electrophoresis |
| TGF-β | Transforming Growth Factor beta |
| TIPS | Thermally induced phase separation |
| TNF-α | Tumor Necrosis Factor Alpha |
| VEGF | Vascular Endothelial Growth Factor |

### BRIEF DESCRIPTION OF THE FIGURES

FIG. 1 is a photographical representation showing a SDS PAGE comparison of Matrigel to various other tissue matrices. (From left to right) Lanes 1 and 2 represent pre-stained molecular weight standards; Lanes 3 and 4 represent commercial Matrigel (BD Biosciences); Lane 5 represents rat muscle matrix; Lane 6 represents pig muscle matrix; Lane 7 represents human muscle matrix. All samples were loaded at a concentration of approximately 10 μg total protein.

FIG. 2 is a photographical representation showing (a) a muscle matrix preparation from the skeletal muscle of a human. Pictures (b) and (c) illustrate the muscle matrix in one of its alternate forms, a sponge formed from TIPS processing of the muscle matrix. Picture (c) is a scanning electron micrograph of the sponge in picture 2(b).

FIG. 3 is a photographical representation of (a) crude pig muscle derived muscle matrix, and (b) Matrigel.

FIG. 4 are micrographic representations showing successful generation of tissue including adipose tissue in the rat. FIGS. 4(a) and (b) are representative sections from a rat tissue engineering chamber model which was coated with MyoGel prior to implantation.

FIG. 5A is a photographic representation showing control of preadipocytes on tissue culture plastic, showing with differentiation. 5B shows preadipocytes on MyoGel, showing lipid accumulation and differentiation towards mature adipocytes. Inset shows a higher magnification picture of a single cell, accumulating lipid. 5C shows a low power micrograph of a mouse chamber that has been filled with muscle extract which has induced adipogenesis.

FIG. 6 is a photographic representation are representative examples of the western blots of various ECM components on different MyoGel species. All arrows and numbers represent MW levels. 6A is directed to immunoblotting for Collagen I on rat muscle extracts, labeling three chains (100, 200, 300 kD). Lanes 1-10 are ten different rat muscle extract preparations. 6B shows laminin α4 (180 kD) and α2 (80 kD fragment and some faint at 300 kD) in rat muscle extracts. Lane 1 is a Matrigel control and lanes 2-11 are the same rat muscle extract preparations, showing strong bands especially for the α4 chain. 6C shows Heparin Sulphate proteoglycans binding in the rat MyoGel HSPG gel lane 1 is a molecular weight standard, lane 2 is Matrigel, with perlecan staining above the 200 kD bar and lanes 3-11 are rat muscle extracts. 6D Fibronectin fragment binding in rat muscle extracts samples, lane 1 is a Matrigel control and lanes 2-11 are the same rat muscle extract.

### DETAILED DESCRIPTION OF THE PREFERRED EMBODIMENTS

The present invention provides a tissue preparation useful, inter alia, for adipogenesis applications and other applications relating to tissue engineering, augmentation, repair and research. The preparation also has applications in the topical therapeutic, cosmetic and food industries. One preferred form of the material comprises solubilized, extracted basement membrane material derived from muscle. Another form comprises intact matrix with lysed cells and basement membrane material. Still another preferred form comprises intact tissue and cells. The tissue material is, therefore, referred to variously as muscle extract material, muscle matrix, myomatrix, myotrix, muscle basement membrane matrix, muscle scaffold, myogel and a cell composition or preparation. These terms are used interchangeably throughout the specification but are encompassed by the term “muscle matrix”.

The muscle matrix preparation, therefore, may be either cell-based or an intact or extracted extracellular matrix. The cell-free extract is generally prepared using methods such as urea or SDS extraction. Intact cellular extract material is generally prepared by freeze/thawing followed by washing, but could include the residue after extraction. Cell-based preparations are generally prepared using techniques such as mincing, gluteraldehyde fixation and/or freeze drying. Other similar methods may be employed and all such methods are encompassed by the present invention. These include critical point drying, cross-linking of proteins using fixatives other than glutaraldehyde, and mechanically disrupting fresh muscle. In addition, there are a variety of pre- or post-extraction techniques which may be employed to further enhance the product or maybe used alone. These include manipulation of the material through various physico-chemical procedures (e.g. milling, pulverization, TIPS, etc.), fixation of tissue after freezing and rinsing and altered washing after freezing. Even the starting tissue can be altered such as using smooth or cardiac muscle. All such variations are within the scope of the present invention.

The preferred tissue material of the present invention generally comprises one or more of, but not limited to, laminin, collagen I, collagen IV, entactin/nidogen, heparan sulfate proteoglycan as well as other components including cytokines and growth factors such as, but not limited to, one or more of EGF, bFGF, NGF, PDGF, IGF-1, TGF-β, VEGF and TNF-α. The tissue material is rich in muscle basement membrane components. The tissue material of the present invention has a range of utilities including the study and engineering of, inter alia, tissues comprising but not limited to adipose, muscle, liver, and pancreas. It also provides a basis for an in vitro bioassay for adipogenic potential of source material, i.e. fat and precursor cells for fat from various sites. The preparations further have applications in the topical therapeutic, cosmetic and food industries.

Accordingly, the present invention provides a composition of matter useful in promoting cell growth including differentiation, proliferation, division and/or morphological changes in a cell or tissue, said composition comprising either a cell-based or cell-free extract of a muscle tissue preparation which preparation provides a source of, but not limited to, laminin, collagen I, collagen IV, entactin/nidogen, heparan sulfate proteoglycan as well as other components including cytokines and growth factors such as, but not limited to, one or more of EGF, bFGF, NGF, PDGF, IGF-1, TGF-β, VEGF and TNF-α or homologs thereof.

In a preferred embodiment, the tissue material comprises laminin, entactin/nidogen, heparan sulfate proteoglycan, collagen IV, bFGF, PDGF, TGF-β, VEGF and TNF-α.

For convenience the term “cell effects” will be used to encompass growth and division of cells differentiation, proliferation and morphological changes.

The source of the tissue material may be from any animal and preferably a mammal such as, but not limited to, a human, non-human primate (eg. gorilla, marmoset or orangoutang), livestock animal (eg. cow, sheep, pig, horse, donkey, goat, camel), laboratory test animal (eg. mouse, rat, rabbit, guinea pigs, hamster) or companion animal (eg. dog, cat). The present invention also extends to avian sources such as chickens, ducks, geese, turkeys and other poultry or game birds, reptilian sources such as snakes and lizards and amphibians sources such as frogs and toads.

In a particularly preferred embodiment, muscle tissue from a pig, mouse, rat or human is used.

Accordingly, another aspect of the present invention provides a composition of matter comprising a muscle preparation from a mammal, said composition comprising:
(i) cell-based or cell-free material;
(ii) components selected from the list comprising one of more of, but not limited to, laminin, collagen IV, entactin/nidogen, heparan sulfate proteoglycan;
(iii) cytokines or growth factors selected from the list comprising one or more of, but not limited to, EGF, bFGF, NGF, PDGF, IGF-1, TGF-β, VEGF and TNF-α or homologs thereof; and
(iv) a total protein content of between from about 1 μg/ml to about 100 mg/ml.

Reference to the above components such as laminin, collagen IV, entactin/nidogen, heparan sulfate proteoglycan or EGF, bFGF, NGF, PDGF, IGF-1, IGF-2, TGF-β, VEGF and TNF-α should be considered as those components but not necessarily restricted to those components, in other words, the preparation may contain other components not recited.

The components of the tissue material may be totally derived from the muscle tissue or additional factors such as, but not limited to, additional gelling agents (such as salts solutes and/or sugars), cytokines, antibiotics, growth enhancers, gene expression enhancers, proliferation inhibitors and/or stem cell differentiation facilitators may be added during preparation.

The tissue material may, in one embodiment, be considered as a composition which facilitates cell culture, cellular differentiation, de-differentiation or growth in vitro or in vivo.

In a particularly preferred embodiment, the tissue material promotes growth and differentiation of cells selected from inter alia stem cells, epithelial cells, skin cells, organ cells and endothelial cells.

Reference to “cell-free material” includes extraction matrix alone or a preparation where cells have been lysed and largely removed.

The present invention further provides a cell culture composition useful in facilitating growth and differentiation of cells or effecting a change in cell or tissue morphology wherein said cell culture composition comprises one or more of, but not limited to, laminin, collagen I, collagen IV, entactin/nidogen, heparan sulfate proteoglycan as well as other components including cytokines and growth factors such as, but not limited to, one or more of EGF, bFGF, NGF, PDGF, IGF-1, IGF-2, TGF-β, VEGF and TNF-α or homologues thereof and which cell culture composition polymerizes into a gel.

Reference to a “cytokine” includes a single or multiple cytokines selected from the list provided. Of course, additional cytokines may be present or included. Likewise, one of laminin, collagen IV, entactin/nidogen and/or heparan sulfate proteoglycan may be present or two or more of these components may be present. Additional extracellular matrix material may also be present or added.

The polymerization generally occurs at temperatures from about 15° C. to about 50° C. such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50° C. Fluctuating temperatures may also be employed.

The term “gel” is used in its broadest sense and includes a semi-liquid, semi-rigid material, flexible material, dense liquid, cream, solid support or combination thereof including a material suitable for use as a food additive.

In a preferred embodiment, the cytokines are present in amounts as follows:
bFGF: from about 0.3 ng/ml to about 4 ng/ml such as 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 ng/ml.
PDGF: from about 1 pg/ml to about 3000 pg/ml such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000 pg/ml.
TGF-β: from about 1 pg/ml to about 2000 pg/ml such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000 pg/ml.
EGF: from about 0 ng/ml to about 100 ng/ml such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 0.96, 97, 98, 99, 100 ng/ml.
VEGF: from about 1 pg/ml to about 3000 pg/ml such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000 pg/ml.
TNF-α: from about 0 pg/ml to about 1000 pg/ml such as 0, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg/ml.

In one embodiment, IGF-1 is absent.

The present invention provides, therefore, tissue-derived material in the form of a cell-based or cell-free preparation useful in cell growth and development and in effecting a change in cell or tissue morphology, said material derived from human non-human primate, livestock animal, companion animal, avian, reptile or amphibian muscle and comprising one or more cytokines and/or growth factors selected from the list comprising, but not limited to, from about 1.0 ng/ml to about 4 ng/ml bFGF, from about 1 pg/ml to about 3000 pg/ml PDGF, from about 1 pg/ml to about 2000 pg/ml TGF-β, from about 1 pg/ml to about 3000 pg/ml VEGF, from about 0 pg/ml to about 1000 pg/ml TNF-α and from about 0 ng/ml to about 100 ng/ml EGF, said material further comprising one or more components selected from, but not limited to, laminin, collagen IV, entactin/nidogen and/or heparan sulfate proteoglycan.

The tissue material is preferably in a gel form with lamellar structures resembling basement membranes and/or components thereof. The tissue material may be in the gel form or it may be in a “precursor” form which is polymerizable to a gel form or it may be cell-based. Conveniently, the tissue material, when in precursor form, is reconstitutable to a gel or matrix form. Even more conveniently, the matrix form of the reconstituted precursor is referred to herein as “muscle matrix”. The muscle matrix of the present invention, either in gel form or precursor form including a cell-based preparation may also be made into or incorporated into beads, sponges, creams and the like. Although a gel form is one preferred form of the preparation, a cell-based preparation which has similar “gelling” characteristics to a gel is also contemplated by the present invention.

Accordingly, the muscle matrix of the present invention is useful in the promotion of cell growth and differentiation of a variety of cells and to effect a change in cell or tissue morphology. Epithelial cells, endothelial cells, neural cells and stem cells are particularly amenable for growth and differentiation by the muscle matrix. It also aids in cell adhesion and in the growth, development, differentiation and/or proliferation of cells selected from, but not limited to, neurons, hepatocytes, Sertoli cells, hair follicles, thyroid cells and the like. As indicated above, a “muscle matrix” is to be understood as covering both a cell-based and cell-free preparation.

Cells may be cultured in vitro on the muscle matrix and then returned to the animal from which they originated or in immune suppressed or histocompatible animals. In this context, an “animal” includes a human, non-human mammal, livestock animal, companion animal or avian, reptilian or amphibian species. Likewise, the muscle matrix may be used alone in vivo to promote cell growth or tissue growth at particular sites or in chambers or other scaffolds implanted in the body.

The muscle matrix of the present invention is generally prepared at a low temperature such as from about 1° C. to about 10° C. for example 1, 2, 3, 4, 5, 6, 7, 8, 9, 10° C. or a non-integral temperature within this range. A temperature of 4° C. is particularly useful. Fresh muscle is collected. From about 10 to about 100 g is convenient to handle. Visible fat is trimmed off the muscle and the tissue is exposed to protease inhibitors in a buffer (eg. NaCl buffer). For preparation of a cell-free extract, the resulting tissue is homogenized and then centrifuged to remove the supernatant. The pelleted tissue is resuspended in a buffer (eg. NaCl) and re-homogenized. The two washing steps are repeated. After the final wash, the pellet is resuspended in a urea buffer. The homogenate is stirred overnight and a series of washes completed, each time collecting the supernatant. The supernatant is then filtered through gauze to remove free floating fat. This is then dialyzed against a solvent (eg. chloroform) overnight to sterilize the material. The solvent is changed to a buffer and dialysis continued to remove the solvent. After the last dialysis, the final buffer used is DMEM or its equivalent. The resulting pre-matrix material is dispensed and stored at −20° C.

When required, a sample of the pre-matrix material is retrieved and incubated at from about 20 to about 50° C. such as around 37-42° C. where the material polymerizes into a gel-like form. Additives to assist gelling can also be added, including but not restricted to plasma.

Extracts may also be prepared using SDS. Alternatively, preparations may be prepared using freeze/thawing followed by washing. This is referred to as intact matrix. Cell-based preparations (i.e. intact tissue) are conveniently prepared using mincing, freeze drying and/or gluteraldehyde fixing.

The tissue extract material of the present invention or muscle matrix may be packaged for sale in a pre-matrix form or in a matrix form and may come with instructions on how to use. Additional components may be in the package or kit and are included prior to use or admixed at the time of use.

As indicated above, the muscle matrix is particularly suited to the culture of a variety of cells including but not limited to adipocytes, 3T3-L1 cells, HUVEC, MCF-7 and MDA-MB-231 breast cancer cells, PC-12 cells and NG-108 neural cells as well as a range of preadipocytes isolated by standard procedures.

The muscle matrix is also useful in an in vitro bioassay for adipogenic potential of source material. The preparation also has ability in in vitro bioassays for the differentiation of other basement membrane-response cell types such as epithelial, neuronal, endothelial and many pathogenic states such as cancer and diabetes.

According to this aspect, the muscle matrix is subjected to an in vitro assay to determine constituent components including cells and/or molecule components which induce adipogenesis. The assay includes coating a surface of a recepticle with a layer or multiple layers of potential adipogenic components to be tested such as an extract of the muscle matrix, seeding cells with a potential to undergo adipogenic differentiation and then screening for adipogenesis. Alternatively, muscle matrix is coated onto the surface and other compounds added and then the system is screened for enhanced or reduced adipogenesis. In another embodiment, cells with a potential to undergo adipogenesis differentiation are maintained in a suspension culture and the media supplemented with a potential adipogenesis component to be tested such as an extract or fraction of muscle matrix. An advantage of cell suspension allows for the rapid isolation of cells from the culturing media to determine if they have undergone differentiation. Alternatively, the assay includes the generation of a three dimensional scaffold comprising a potential adipogenic component or extract, seeding cells with a potential to undergo adipogenic differentiation and then screening for adipogenesis. Thus, the assay system of the present invention may also have the added advantage of providing or selecting or developing optimised populations of pre-adipocytes for use in tissue engineering. The assay may conveniently be conducted in a suitable receptacle in vitro. In this case, the surface of the matrix may be coated with a cell material preparation. The receptacle may also be packaged for sale with instructions for use.

Accordingly, the present invention contemplates an in vitro assay for adipogenesis modulating components, extracts, or cell systems, said assay comprising screening a muscle preparation to identify a group of cells having a propensity for adipocytic differentiation, generating or obtaining a potential adipogenesis modulating component or extract or cell system, seeding onto said component or extract said group of cells having a propensity for adipocytic differentiation, incubating said cells for a time sufficient for adipogenesis to occur and then screening said cells for adipocytic differentiation. A “cell system” in this context has the same meaning as a cell-based preparation.

In some embodiments the potential adipogenesis modulating component or extract or cell system promotes adipogenesis. In other embodiments, the adipogenesis modulating component or extract or cell system inhibits adipogenesis.

By “modulating” is meant increasing or decreasing, either directly or indirectly the level of adipogenesis.

A layer of potential adipogenesis modulating component or extract or cell system may be obtained or generated. Alternatively, a three-dimensional support matrix comprising the potential adipogenesis modulating component or extract or cell system may be obtained or generated. In certain embodiments the cells having a propensity to undergo adipocytic differentiation may be maintained in a suspension culture and the media is supplemented with the potential adipogenesis modulating component or extract. Reference to a “layer” includes two or more layers. The present method extends to adding potential adipogenesis promoting agents to muscle matrix.

Yet another aspect of the present invention provides a method of generating donor vascularized tissue suitable for transplantation into a recipient, said method comprising creating a vascular pedicle comprising a functional circulatory system and having tissue or tissue extract or a component thereof impregnated, attached or otherwise associated with the vascular pedicle; associating the vascular pedicle within and/or on a support matrix; seeding the support matrix with isolated cells or pieces of tissue identified using an in vitro assay as promoting adipogenesis; or some other useful endpoint implanting the support matrix containing the vascular pedicle into a recipient at a site where the functional circulatory system is anastomosized to a local artery or vein; and leaving the support matrix at the implantation site for a period sufficient to allow the growth of vascularized new tissue wherein the impregnated material or seeding material is selected on a particular basis, for example, that it promotes adipogenesis when determined by the assay comprising screening a tissue or tissue extract to identify a group of cells having a propensity for adipogenic differentiation, generating or obtaining potential adipogenesis promoting component or extract, seeding onto said component or extract a group of cells having a propensity for adipocytic differentiation, incubating said cells for a time sufficient for adipogenesis to occur and then screening said cells for adipocytic differentiation.

In a preferred embodiment, the vascular pedicle comprises attached fat or other adipose tissue or tissue comprising myoblasts, fibroblasts, pre-adipocytes and adipocytes, cardiomyocytes, keratinocytes, endothelial cells, smooth muscle cells, chondrocytes, pericytes, bone marrow-derived stromal precursor cells, embryonic, mesenchymal or haematopoietic stem cells, Schwann cells and other cells of the peripheral and central nervous system, olfactory cells, hepatocytes and other liver cells, mesangial and other kidney cells, pancreatic islet β-cells and ductal cells, thyroid cells, cells of other endocrine organs and spheroids of aforementioned cells. All these cells are tested in vitro for their potential to grow/survive on the matrix or capacity to differentiate into other useful tissues e.g. adipogenic potential, prior to selection. The presence of the attached tissue on the vascular pedicle further facilitates the growth of new fat tissue in or around the support matrix. In an alternative embodiment, tissue extract or a recombinant, synthetic or purified component of the tissue is associated with the vascular pedicle. For example, and in a preferred embodiment, these components and extracts are derived from matrix material and screened in vitro for adipogenic potential.

The matrix is allowed to set and cells capable of adipocytic differentiation plated over the monolayer of matrix in the presence of complete media (such as DMEM containing FCS) or differentiation media (complete media supplemented with 1 μM dexamethasone, insulin, indomethacin and IBMX). Adipogenesis is observed over a period of 14 days. Examples of suitable adipocytic cells include 3T3-L1 cells or preadipocytic cells isolated by standard procedures.

The present invention contemplates, therefore, an in vitro assay for adipogenesis promoting components or extracts, said assay comprising generating or obtaining a layer of potential adipogenesis extract from muscle matrix on the surface of a receptacle, seeding onto said layer a group of cells having a propensity for adipocytic differentiation incubating said cells for a time sufficient for adipogenesis to occur and then screening said cells for adipocytic differentiation.

In another embodiment, the assay is conducted on a three-dimensional support matrix, which may be constructed substantially from the muscle matrix or comprise a scaffold that is coated with the muscle matrix, in respect of which three dimensional cell culturing techniques known to the person skilled in the art are carried out, for example the spinner flask technique (Mueller-Klieser J Cancer Res Clin Oncol 13: 101-122, 1986), the liquid-overlay technique (Yuhas, et al., Cancer Res 37: 3639-3643, 1977). Another example of a three-dimensional cell culture technique is a rotating culture vessel specifically engineered to randomize the gravity vector by rotating a fluid-filled culture vessel about a horizontal axis while suspending cells and cell aggregates with minimum fluid shear. These devices have been described in U.S. Pat. Nos. 5,153,131; 5,153,132; 5,153,133; 5,153,034, and 5,155,035.

An advantage of the three-dimensional matrix is that it sustains active proliferation of cells in culture for longer periods of time than will monolayer systems. This may be in part due to the increased area of the three dimensional matrix which results in a prolonged periods of active proliferation of cells. The matrix provides the support, growth factors and regulatory factors necessary to sustain long-term active proliferation of cells in culture. The growth of the cells in the presence of the support may be further enhanced by adding proteins, glycoproteins, glycosaminoglycans, a cellular matrix and other materials to the support itself or by coating the support with these materials. The three-dimensionality of the matrix allows for the formation of microenvironments conducive to cellular maturation and migration. When grown in this three-dimensional system, the proliferating cells mature and segregate properly to form components of adult tissues analogous to counterparts in vivo.

In order for the three dimensional structures to be able to maintain the activity of living cells three dimensional matrices should demonstrate appropriate spatial and compositional properties. Such matrices include hydrogels, or porous matrices such as fibre-based or sponge-like matrices. Common materials used in three-dimensional matrices are natural polymers or “biomatrices”, synthetic polymers and inorganic composites. In the present embodiment, where the method contemplates the use of the three-dimensional matrices for an in vitro assay, the biocompatibility of the matrix is not particularly important.

Preferred examples of biomatrices are those extracted from or resembling muscle matrix or having a cell system comprising same.

The present invention further contemplates the use of muscle matrix in the manufacture of a cell growth promoting composition. The muscle preparation of the present invention is also useful for the selective purification of specific cell types (e.g. preadipocytes) for complex cell mixtures based on selected growth and morphological characteristics specific to the muscle preparation.

The articles “a” and “an” are used herein to refer to more than one (ie. to at least one) of the grammatical object of the article. By way of example, “an component” means one component or more than one component.

The present invention is further described by the following non-limiting Examples.

### EXAMPLE 1

### Matrix Extraction-I

Muscle samples were collected either from freshly sacrificed animals (rat and pig) or from patients undergoing reconstructive surgery. All samples were collected under the appropriate ethical committee approval and with fully informed consent. All steps of the procedure were performed on ice or at 4° C.

Muscle was collected, weighed and trimmed of fat prior to matrix extraction. Samples were then washed and homogenized in ice cold 3.4M NaCl buffer to which was added protease inhibitors (0.5 mM PMSF, 2 mM EDTA, 0.1M EACA, 2 mM NEM). The homogenate is then centrifuged at 10,000 rpm at 4° C. for 15 minutes, following which the supernatant is discarded and pellets are resuspended in the 3.4M NaCl buffer. This step is repeated 2-3 times.

Pellets are then resuspended in a 2M urea buffer at an equivalent volume to the original volume of the tissue, homogenized and stirred overnight at 4° C. Following this, the extract is centrifuged at 14,000 RPM at 4° C. for 30 minutes and the supernatant reserved. Pellets are re-homogenized in half the original volume of 2M urea buffer then the centrifuge step is repeated. The supernatant is then combined with the previously reserved supernatant and filtered.

The extract is then dialyzed against 0.5% v/v chloroform in 0.05M Tris/0.15M NaCl buffer (TBS) at 4° C. overnight as a sterilization step. The extract is then dialyzed against several changes of TBS alone, followed by DMEM. Aliquoted extract was stored at −20° C.

### EXAMPLE 2

### Matrix Extraction-II

All steps were performed at 4° C. or on ice. Muscle tissue was collected as fresh as possible (about 20-30 gms minimum preferably) and weighed. All visible fat was trimmed from muscle as quickly as possible in steel dissecting tray. Cold 3.4M NaCl buffer was added with protease inhibitors at a 2:1 volume ratio (eg. 100 ml buffer to 50 gm of muscle) to a beaker on ice and muscle added. The sample was then homogenized thoroughly. The muscle homogenate was centrifuged at 10,000 RPM at 4° C. for 15 minutes. Supernatant was then discarded and pellets were resuspended in the same amount of 3.4M NaCl buffer and re-homogenized. This step was repeated twice making for a total of 3-4 washes in NaCl. After the third wash the pellets are resuspended and homogenized in a 1:1 volume of 0.5M NaCl in 50 mM Tris-HCl (pH 7.4) with protease inhibitors (eg. 50 ml to 50 mg of muscle). The sample was spun overnight at 4° C. using a magnetic stirrer. The sample was then centrifuged for 30 minutes at 14,000 RPM at 4° C., the supernatant removed and the sample stored. The pellet was then homogenized in 2.0M Guanadine hydrochloride in 50 mM Tris-HCl (pH 7.4) with 0.2 mM dithoithreitol. The homogenate was stirred overnight and centrifuged for 30 minutes at 14,000 rpm at 4° C. and the supernatant removed and stored.

All steps following were performed on the two supernatants, which were kept separate throughout these steps.

Supernatant was filtered through gauze to remove free floating fat, etc. The extract was dialyzed against 1-2 litres of 0.2% v/v chloroform in TBS buffer (5 mls/litre) overnight on a magnetic stirrer at 4° C. This was a sterilization step. The buffer was changed for clean TBS and dialyzed for at least 8 hours. This step was repeated three times. When the dialysis buffer was changed the tubing end was rotated several times to ensure mixing. After the last TBS dialysis exchange the buffer for DMEM and dialyze overnight. Thicker solutions such as pig muscle matrix require longer. These two extracts are then mixed together to give matrices of different configurations in order to improve gelation. The samples were then Aliquoted into sterile test tubes. This was performed on ice and under a flow cabinet. The samples were then stored at −20° C.

### EXAMPLE 3

### SDS-PAGE

Protein concentration was measured by bicinchoninic acid (BCA protein assay kit). Matrix samples were prepared at 0.5-1.0 mg/mL in Laemmli solution (Laemmli, Nature 227:680-685, 1970) and boiled for 5 minutes prior to resolving on SDS-PAGE gels. Sample volumes of 15 μL were loaded in lanes and separated by SDS-polyacrylamide gel electrophoresis on a 4-12% w/v gradient polyacrylamide gel (Invitrogen). Gels were run for 50 minutes at a constant voltage of 200V, after which the gels were removed and either stained with Coomassie Brilliant Blue or transferred for immunoblot analyses.

### EXAMPLE 4

### Immunoblot Analyses

Proteins were resolved on SDS-PAGE gels as described in Example 3 and unstained gels were transferred to nitrocellulose sheets. Transfer was following the wet transfer procedure by applying a constant voltage of 100V for 1 hour, current commenced at 220 mA. Blots were first incubated in 5% w/v non-fat dried milk in Tris buffered saline (TBS) containing 0.1% v/v Tween 20 (TTBS) overnight to reduce non-specific reactions. Primary antibodies were incubated for 1 hour at room temperature, rinsed three times with TTBS and then incubated for 1 hour with a peroxidase conjugated secondary antibody (1:5000-1:10000). Blots were rinsed again for three times in TTBS before the immunoreactive proteins were visualized by using enhanced chemiluminescence Western blotting detection system (Amersham Pharmacia). Six primary antibodies were used: HSPG 1:5000 (Seikagaku Corporation), Laminin 1:5000 (Dako), Nidogen 1:10000 (Chemicon), and Collagen IV 1:10000 (Dako), Fibronectin 1:10 000 and SPARC 1:10 000

### EXAMPLE 5

### Analysis of Muscle Matrix

An analysis of the components of muscle matrix compared to the preparation from BD Biosciences (Matrigel) was conducted and the results are shown in Tables 2, 3 and 4.

Characterization of Growth Factors:

Growth factor/cytokine levels were measured using Quantikine ELISA kits (R&D Systems) following kit instructions. Growth factors tested included vascular endothelial cell growth factor (VEGF), platelet derived growth factor (PDGF), transforming growth factor beta (TGF-β), basic fibroblast growth factor (bFGF), tumor necrosis factor alpha (TNF-α), epidermal growth factor (EGF), insulin-like growth factor 1 (IGF-1), leukemia inhibitory factor (LIF) and nerve growth factor (Chemicon kit) (NGF). Briefly, the extracts were diluted (1:5-1:10) and incubated with standards and controls in an antibody coated plate for 2-3 hours. Plates were washed and, a polyclonal HRP-conjugated secondary antibody added. Following incubation, excess conjugate was removed and the plate incubated a third time with a colour substrate. After the addition of a stop solution, plates were read using a microplate reader (Axion, Mutliskan, USA) set at λ450 nm absorbance with a correction reading set at λ 550 nm. All measurements and calculations were performed using Genesis 2.0 plate reading software (details) and values were translated to pg/mg of matrix. Table 2 contains the growth factor levels in Matrigel (reg trademark) as reported by the manufacturer and as measured by ELSA.

TABLE 2

| | BD Full | | BD GRF | | BD Full | | BD GFR | | KK Full | | KK GFR | |
|---|---|---|---|---|---|---|---|---|---|---|---|---|
| | (spec sheet) | | (spec sheet) | | (ELISA) | | (ELISA) | | (ELISA) | | (ELISA) | |
| bFGF | 0.1 | pg/ml | 0.1 | pg/ml | 48 | pg/ml | — | | 304 | pg/ml | 0 | pg/ml |
| PDGF | 12 | pg/ml | <5 | pg/ml | 0 | pg/ml | 0 | pg/ml | 12 | pg/ml | 0 | pg/ml |
| TGFβ | 2.3 | ng/ml | 1.7 | ng/ml | 2.3 | ng/ml | 1.8 | ng/ml | 4.5 | ng/ml | 1.6 | ng/ml |
| EGF | 0.5-1.3 | ng/ml | <0.5-1.3 | ug/ml | 0 | ng/ml | 0 | ng/ml | 0 | ng/ml | 0 | ng/ml |

| | | | | | | | | | | |
|---|---|---|---|---|---|---|---|---|---|---|
| VEGF | NR | NR | 8.2 | ng/ml | 2.9 | ng/ml | 8.3 | ng/ml | 0.8 | ng/ml |
| TNFα | NR | NR | 0 | pg/ml | 0 | pg/ml | 0 | pg/ml | 0 | pg/ml |

BD Full = Commercial Matrigel (reg trademark)
BD GFR = Growth factor reduced Matrigel (reg trademark)
KK Full = Our own Matrigel (reg trademark) preparation
KK GFR = Our own growth factor reduced preparation
NR = Not recorded

Tables 3 and 4 contain the growth factor levels in muscle matrices made from rat, pig and human as measured by ELISA. Table 3 represents the range of values. Table 4 represents the mean and SEM of the data.

TABLE 3

| Growth | Rat | | Human | Mouse | |
|---|---|---|---|---|---|
| factor | extract | Pig extract | extract | extract | Matrigel |
| (pg/mg) | (n = 10) | (n = 10) | (n = 6) | (n = 3)## | (n = 3-7) |
| FGF2 | 27-297 | 14-1281 g# | 41-250 | | 0-18 |
| VEGF | 7-64 | NM | NM | 34-45 | 801-1703 |
| PDGF | 2-45 | 1-8 | 5-12 | NM | 0-2.7 |
| NGF | 2-100 | 13-22 | 18-71 | NM | |
| TGF-β | 0-9 | NB | 0-45 | NM | 32-88 |
| TNFα | 3-21 | 3-12 | 2-11 | NM | 2-6 |
| EGF | NB | NB | 2-18 | NM | |
| LIF | NB | NB | NB | 3-9 | |

TABLE 4

| | Human | Pig | Rat | | Matrigel |
|---|---|---|---|---|---|
| Matrix source | (n = 6) | (n = 10) | (n = 10) | Mouse (n = 3) | *** |
| bFGF | 131.16 ± 36 | 217.9 ± 121 | 113.5 ± 32.6 | | 6.17 ± 2.3 |
| VEGF | 1.05 ± 0.4 | 0.58 ± 0.25 | 23.15 ± 5.8 | 41.3± | 1145 ± 165 |
| TGF-β | 24.75 ± 8 | NB | 1.7 ± .94 | | 68.5 ± 17.8 |
| TNF-α | 7.38 ± 1.3 | 7.15 ± 1.14 | 7.1 ± 1.7 | | 2.8 ± 1.7 |
| PDGF | 7.90 ± 0.8 | 3.50 ± 0.6 | 10.56 ± 4.1 | | 0.9 ± 0.9 |
| NGF | 33.9 ± 7.9 | 18.2 ± 1.9 | 28.5 ± 9.1 | | 15.03 |
| EGF | 9.3 ± 2.4 | NB | NB | | NB |
| LIF | NB | NB | NB | | 56.7 |

# = one very high reading - all others below 339 pg/mg.
## these samples were run to check and see if cross species binding was an issue
NB: No binding observed in these samples observed using these ELISAs
NM: Not measured

Total Protein Concentration Measurements

The total protein levels of matrices were measured using a Bicinchoninic Acid assay (Amersham Biosciences Corporation New Jersey, USA). Samples were measured at a 1:10 dilution and assayed in accordance with the kit instructions against a series of BSA standards. After incubation samples were analysed using a spectrophotometer t at 562 nm wavelength. Ten samples each were analysed for rat and pig muscle matrix, and six for human muscle matrix. Table 5 shows the total protein levels in muscle matrices and Matrigel (reg trademark) as measured by BCA total protein assay

TABLE 5

| | Matrix Source | | | |
|---|---|---|---|---|
| | Pig Muscle | Rat Muscle | Human Muscle | Matrigel |
| Total Protein | 4-15 mg/ml | 6-12 mg/ml | 4-10 mg/ml | 8-10 mg/ml |

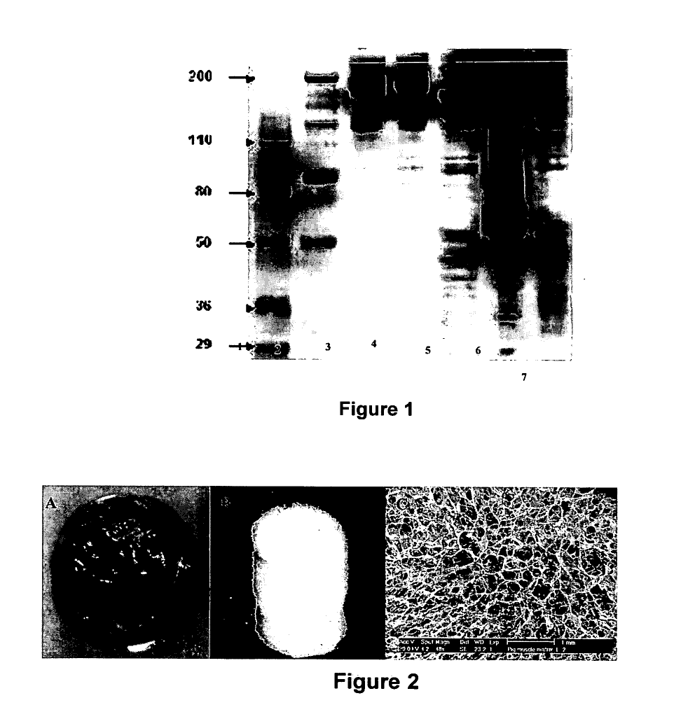

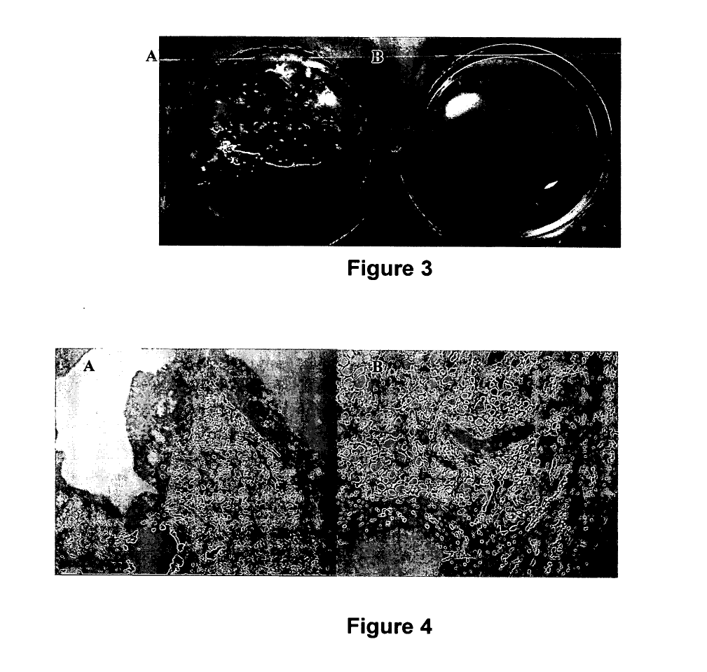

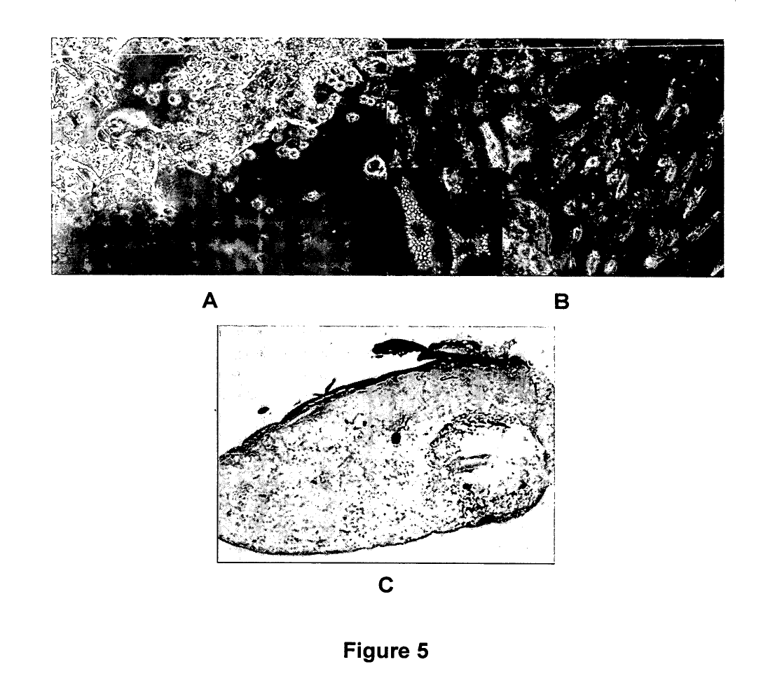

The muscle matrix is also described in FIGS. 1 through 4. FIG. 1 provides an SDS-PAGE comparison of Matrigel with other tissue matrices. FIG. 2 is a photographic representation of muscle matrix from skeletal muscle. FIG. 3 is a photograph of pig muscle-derived muscle matrix. FIG. 4 is a micrograph showing the successful generation of tissue in rat. FIG. 5.

Measurement of ECM Components

1,9-dimethylmethylene blue, Direct Red 80 (Sirius Red), chondroitin sulfate A, papain, dithiothreitol and collagen I were all purchase from Sigma-Aldrich. Sulfated glycosaminoglycans (GAGs) were quantified in MyoGel samples by precipitation with the dye 1,9-dimethylmethylene blue (DMMB). Interfering proteins were digested by the addition of an equal volume of 40 mM sodium phosphate buffer (pH 6.8) containing 0.6 mg/ml papain, 2 mM EDTA and 4 mM DTT, followed by incubation at 60° C. for 60 min. (Farndale et al. Biochimica ET Biophysica Acta 882:173-177, 1986). Aliquots (100 uL) of each sample were then incubated with 1 mL of DMMB solution (16 mg/L DMMB in 0.2 M GuHCl, 1 g/L sodium formate and 1 ml/L formic acid) for 30 min., and mixed continuously on a rotating wheel. Following precipitation of the GAG-DMMB complex, the insoluble material was separated from the supernatant by centrifugation (10,000×g for 10 min) and the supernatant was removed. The dye was liberated from the pellet by the addition of 1 mL of decomplexation buffer (50 mM sodium acetate buffer, pH 6.8, containing 10% propan-1-ol and 4M GuHCl) (Barbosa et al. Glycobiology 13:647-653, 2003). The absorbance (λ 650 nm) of the buffer was then determined in a microplate reader (Multiskan RC; Labsystems). Chondroitin sulfate A was employed as a standard.

Laminins were estimated following their elution from a 1 mL Heparin affinity column (Amersham Biosciences; Uppsala, Sweden) (Talts et al. EMBO J. 18:863-870, 1999), (Talts et al. J Biol Chem 275:35192-35199, 2000). In brief, MyoGel samples were solubilised by incubation with an equal volume of 4M GuHCl+2 mM DTT in 50 mM Tris-HCl (pH 7.4), for 24 hours at 4° C. Following overnight dialysis against 50 mM Tris-HCl (pH 7.4)+0.15M NaCl, a 1 mL aliquot was applied to the affinity column. Non-laminin proteins were washed from the column with 5 mL of 50 mM Tris-HCl (pH 7.4)+0.15M NaCl, and the laminins were eluted with 5 mL of 0.5M NaCl in 50 mM Tris-HCl (pH 7.4). Laminin was then estimated with the Bio-Rad Protein Assay using the microplate microassay procedure as described above

Collagens were determined via their precipitation by the polyazo dye Sirius Red (Marotta and Martino Analytical Biochemistry 150:86-90, 1985). An aliquot of MyoGel (100 μL) was incubated with 1 mL of 50 μM Sirius Red in 0.5 M acetic acid, for 30 min at room temperature. Following centrifugation (10,000×g for 10 min), the absorbance (λ 550 nm) of the supernatants were determined in a microplate reader. Collagen I from rat tail was used as a standard.

Hyaluronan levels were measured by an ELISA (Echelon, UT, USA) following kit instructions.

Table 6 and 7 show extracellular matrix components in muscle matrices made from rat, pig and human. Table 6 represents the range of values, while Table 7 represents the mean and SEM of the data.

TABLE 6

| | Matrix | | |
|---|---|---|---|
| | Rat extract | Pig extract | Human extract |
| GAG | 0-5 μg/mg | 0.5-22 μg/mg | 0.6-1.8 μg/mg |
| Laminin | 223-700 μg/mg | 62-1000 μg/mg | 375-1000 μg/mg |
| Collagen | 49-532 μg/mg | 45-1000 μg/mg | 54-1249 μg/mg |
| Hyaluronan | 5-10 ng/mg | 0-5 ng/mg | 0-5 ng/mg |

TABLE 7

| | Human extract | Pig extract | Rat extract | percentage |
|---|---|---|---|---|
| μg/mg Matrix | | | | |
| GAG | 0.86 ± 0.2 | 6 ± 2.8 | 2.45 ± 0.6 | 0.4% |
| Laminin | 865.57 ± 150 | 442 ± 120 | 422.8 ± 67 | 45% |
| Collagen | 437.61 ± 175 | 486.75 ± 133 | 254.28 ± 46.7 | 27% |
| ng/mg matrix | | | | |
| HYALURONAN | 5-10 | <5 | <5 | NS |

SDS-PA GE/Immunoblot Assays

Matrix samples were prepared at 0.5-1.0 mg/mL in Laemmi solution (Laemmli, 1970,) and boiled for 5 minutes prior to resolving on SDS-PAGE gels. Samples volumes of 10 μL were loaded in lanes and separated by SDS-polyacrylamide gel electrophoresis on either a 3-8% or 4-12% gradient polyacrylamide gel (Invitrogen, Carlsbad, Calif., USA). Gels were run for 45 minutes at a constant voltage of 200V, after which the gels were removed and either stained with Coomassie Brilliant Blue or transferred for immunoblot analyses.

For immunoblots, proteins were resolved on SDS-PAGE gels are described above and unstained gels were transferred to nitrocellulose sheets. Transfer was by wet transfer procedure, applying a constant voltage of 30V for 1 hour. After transfer, blots were incubated in 5% non-fat dried milk (Homebrand, Safeways AUS] in phosphate buffered saline (PBS) containing 0.1% Tween 20 (TPBS) overnight to reduce non-specific binding. Blots were incubated in primary antibodies for 1 hour at room temperature, rinsed three times with TPBS and then incubated for 1 hour with an infrared labelled secondary antibody (Molecular Probes, UT, USA or Rocklands, CA, USA) appropriate to the primary used (1:10 000). Blots were rinsed again three times in TPBS before the immunoreactive proteins were scanned into the Odyssey Infrared detection system (Licor Biosciences, USA). Primary antibodies used included anti-HSPG 1:5000 (Seikagaku Corporation, Japan), Laminin α4 and α2 1:1000 (kind gift of Dr Lydia Soroken), Nidogen 1:3000 (Chemicon, USA), Fibronectin 1:5000, Collagen 1:1:10000, Collagen IV 1:10000 and SPARC 1:10 000 (kindly supplied by Dr H Kleinman, NIH USA).

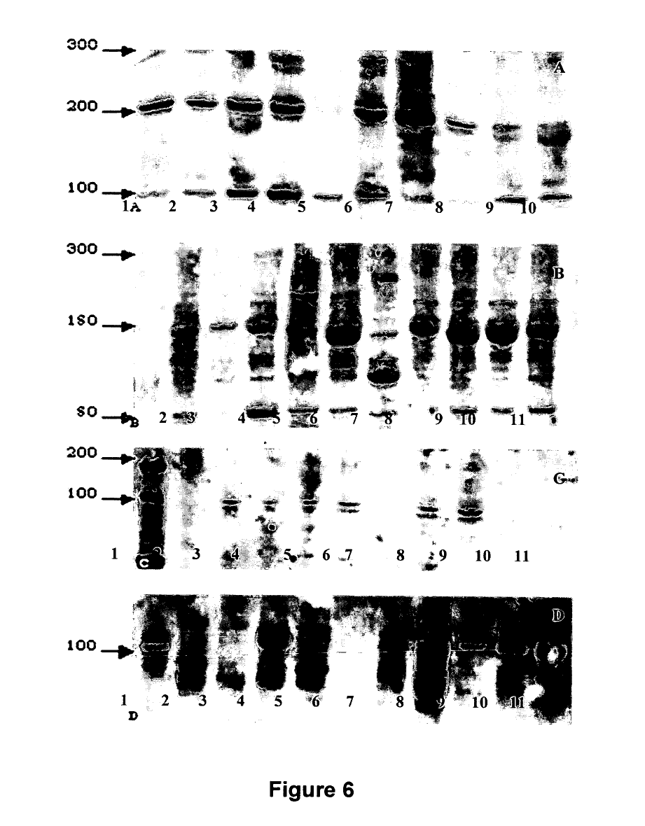

FIG. 6 are representative examples of the western blots of various ECM components on different MyoGel species. All arrows and numbers represent MW levels. 6A is directed to immunoblotting for Collagen I on rat muscle extracts, labeling three chains (100, 200, 300 kD). Lanes 1-10 are ten different rat muscle extract preparations. 6B shows laminin α4 (180 kD) and α2 (80 kD fragment and some faint at 300 kD) in rat muscle extracts. Lane 1 is a Matrigel control and lanes 2-11 are the same rat muscle extract preparations, showing strong bands especially for the α4 chain. 6C shows Heparin Sulphate proteoglycans binding in the rat MyoGel HSPG gel lane 1 is a molecular weight standard, lane 2 is Matrigel, with perlecan staining above the 200 kD bar and lanes 3-11 are rat muscle extracts. 6D Fibronectin fragment binding in rat muscle extracts samples, lane 1 is a Matrigel control and lanes 2-11 are the same rat muscle extract.

In Vitro Cell Differentiation Assays

The assays were developed using rat epididymal preadipocytes. The assays were performed in 24-well plates for morphological analyses. 300 μl of extracellular matrix (ECM)/well was added to 24-well plates. The matrices were set at 37° C. for 20-30. Following this, cells were added to each well (0.3×106 cells/well for 24-well culture plates). Cells were allowed to adhere to the matrices overnight at 37° C./5% CO₂. Differentiation was observed over a period of 14 days with photographs taken every 4-5 days. Cells seeded onto tissue culture plastic alone were used as controls.

FIG. 5 shows control preadipocytes on tissue culture plastic, showing no differentiation. 5B shows preadipocytes on MyoGel, showing lipid accumulation and differentiation towards mature adipocytes. Inset shows a higher magnification picture of a single cell, accumulating lipid. 5C shows a low power micrograph of a mouse chamber that has been filled with muscle extract which has induced adipogenesis.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Barbosa et al. Glycobiology 13:647-653, 2003
Farndale et al. Biochimica ET Biophysica Acta 882:173-177, 1986
Inoue et al., J Cell Biol, 97:1524-1539, 1983
Kleinman et al., Biochem 21:6188-6193, 1982
Laemmli, Nature 227:680-685, 1970
Marotta and Martino Analytical Biochemistry 150:86-90, 1985
Mueller-Klieser, J Cancer Res Clin Oncol 13: 101-122, 1986
Talts et al. EMBO J. 18:863-870, 1999
Talts et al. J Biol Chem 275:35192-35199, 2000
Terranova et al., Cell 22:719-726, 1980
Yuhas, et al., Cancer Res 37: 3639-3643 1977

What is claimed is:

1. A composition of matter useful in promoting cell effects, said composition comprising:
a) a cell-based or cell-free extract of a muscle tissue preparation;
b) at least one component selected from the group consisting of: laminin, collagen I, collagen IV, entactin/nidogen, and heparan sulfate proteoglycan; and
c) at least one component selected from the group consisting of: EGF, bFGF, NGF, PDGF, IGF-1, TGF-β, VEGF, TNF-α and homologs thereof.

2. The composition of claim 1 wherein the composition comprises laminin, entactin/nidogen, heparan sulfate proteoglycan, collagen IV, bFGF, PDGF, TGF-β, VEGF and TNF-α.

3. The composition of claim 1 wherein the cells are from muscle tissue.

4. The composition of claim 3 wherein the muscle tissue is from an organism selected from the group consisting of a: human, non-human, primate, livestock animal, laboratory test animal, companion animal, avian species, reptile and amphibian.

5. The composition of claim 3 wherein the muscle tissue is from a human, pig or rat.

6. The composition of claim 1 wherein the composition is a cell-based preparation.

7. The composition of claim 1 wherein the composition comprises intact extracellular matrix.

8. The composition of claim 1 wherein the composition comprises extracted extracellular matrix.

9. The composition of claim 1 wherein the composition comprises a total protein content of from about 1 μg/ml to about 100 mg/ml.

10. The composition of claim 1 wherein the composition polymerizes into a gel.

11. The composition of claim 1 further comprising one or more components selected from the group consisting of: exogenous cytokines, antibiotics, growth enhancers, gene expression enhancers, proliferation inhibitors and stem cell differentiation facilitators.

12. The composition of claim 1 wherein said composition promotes effects on cells and wherein said cells are selected from the group consisting of: stem cells, epithelial cells, skin cells, organ cells and endothelial cells.

13. The composition of claim 1 wherein bFGF is present from about 0.3 ng/ml to about 4 ng/ml; PDGF from about 1 pg/ml to about 3000 pg/ml; TFG-β from about 1 pg/ml to about 2000 pg/ml; EGF from about 0 ng/ml to about 100 ng/ml; VEGF from about 1 pg/ml to about 3000 pg/ml; and TNF-α from about Opg/ml to about 1000 pg/ml.

14. A method of manufacturing a bioassay for adipogenic potential of a material comprising:
obtaining a potential adipogenesis promoting component or extract;
seeding onto said component or extract a group of cells having propensity for adipocytic differentiation;
incubating said cells for a time sufficient for adipogenesis to occur; and
screening said cells for adipocytic differentiation.

15. The method of claim 14 wherein said potential adipogenesis promoting component or extract is the composition comprising:
a) a cell-based or cell-free extract of a muscle tissue preparation;
b) at least one component selected from the group consisting of: laminin, collagen I, collagen IV, entactin/nidogen, and heparan sulfate proteoglycan; and
c) at least one component selected from the group consisting of: EGF, bFGF, NGF, PDGF, IGF-1, TGF-β, VEGF, TNF-α and homologs thereof.

16. A method of generating donor vascularized tissue suitable for transplantation into a recipient, said method comprising:
creating a vascular pedicle comprising a functional circulatory system and having tissue or tissue extract or a component thereof impregnated, attached or otherwise associated with the vascular pedicle;
associating the vascular pedicle within and/or on a support matrix;
seeding the support matrix with isolated cells or pieces of tissue identified using an in vitro assay as promoting adipogenesis or some other useful endpoint;
implanting the support matrix containing the vascular pedicle into a recipient at a site where the functional circulatory system is anastomosized to a local artery or vein; and
leaving the support matrix at the implantation site for a period sufficient to allow the growth of vascularized new tissue, wherein the impregnated material or seeding material is selected on a particular basis, for example, that it promotes adipogenesis when determined by the assay comprising: screening a tissue or tissue extract to identify a group of cells having a propensity for adipogenic differentiation, generating or obtaining potential adipogenesis promoting component or extract, seeding onto said component or extract a group of cells having a propensity for adipocytic differentiation, incubating said cells for a time sufficient for adipogenesis to occur and then screening said cells for adipocytic differentiation.

17. The method of claim 16 wherein the vascular pedicle comprises attached fat or other adipose tissue or tissue comprising at least one cell selected from the group consisting of: myoblasts, fibroblasts, pre-adipocytes and adipocytes, cardiomyocytes, keratinocytes, endothelial cells, smooth muscle cells, chondrocytes, pericytes, bone marrow-derived stromal precursor cells, embryonic, mesenchymal cells, haematopoietic stem cells, Schwann cells, other cells of the peripheral and central nervous system, olfactory cells, hepatocytes, other liver cells, mesangial cells, other kidney cells, pancreatic islet β-cells, ductal cells, thyroid cells, cells of other endocrine organs and spheroids of aforementioned cells.

18. The method of claim 17 wherein the vascular pedicle comprises an in vitro assay for adipogenesis promoting components or extracts, said assay comprising generating or obtaining a layer of potential adipogenesis extract from muscle matrix on the surface of a receptacle, seeding onto said layer a group of cells having a propensity for adipocytic differentiation incubating said cells for a time sufficient for adipogenesis to occur and then screening said cells for adipocytic differentiation.
